(19) United States
(12) Patent Application Publication
(10) Pub. No.: US 2020/0085305 A1
(43) Pub. Date: Mar. 19, 2020

(54) COMPOSITIONS, METHODS AND SYSTEMS FOR IDENTIFYING THE POSITION AND ORIENTATION OF THE ESOPHAGUS IN ATRIAL FIBRILLATION ABLATION PROCEDURES

(71) Applicant: Saumil R. OZA, Jacksonville, FL (US)

(72) Inventor: Saumil R. OZA, Jacksonville, FL (US)

(21) Appl. No.: 16/572,498

(22) Filed: Sep. 16, 2019

Related U.S. Application Data
(60) Provisional application No. 62/732,543, filed on Sep. 17, 2018.
(60) Provisional application No. 62/858,128, filed on Jun. 6, 2019.

(51) Int. Cl. A61B 5/00; A61B 18/14; G06T 7/00

(52) U.S. Cl. CPC A61B 5/0044; A61B 18/1492; G06T 2207/30048; G06T 2207/10064; G06T 7/0012

(57) ABSTRACT

The present invention is directed to compositions, methods and systems for identifying, in real time, the position and orientation of the esophagus prior to and during atrial fibrillation ablation procedures, so as to avoid or reduce the incidence of atrioesophageal fistula (AEF). The compositions, methods and systems of the present invention include the identification and visualization of the esophagus, the rapid and accurate integration of the visualized esophagus into an anatomical map together with the posterior wall of the left atrium, in each case presented as a 3-D map, so as to facilitate the accurate identification of those areas of the esophagus that lie in contact with or in near proximity to those areas of the posterior wall of the left atrium that the operator intends to ablate.

### PRIORITY CLAIM

To the fullest extent permitted by law, the present non-provisional patent application claims priority to, and the full benefit of, U.S. Provisional Patent Application No. 62/732,543, filed on Sep. 17, 2018, and entitled “Novel Method for Esophageal Imaging Using Intra-Cardiac Echocardiography During Atrial Fibrillation Ablation”; and, U.S. Provisional Patent Application No. 62/858,128, filed on Jun. 6, 2019, and entitled “Compositions, Methods, and Systems for Identifying the Position and Orientation of the Esophagus in Atrial Fibrillation Ablation Procedures,” the contents of each of which are incorporated herein by reference as if reproduced in their entirety.

### FIELD OF THE INVENTION

The present invention relates generally to atrial fibrillation ablation procedures, and more specifically to compositions, methods and systems for identifying the position and orientation of the esophagus relative to the left atrium prior to and during atrial fibrillation ablation procedures.

### BACKGROUND OF THE INVENTION

Atrial fibrillation (or “AFib” or “AF”) is a common form of heart arrhythmia that occurs when the upper chambers or atria of the heart beat irregularly. Left untreated, AFib can lead to heart-related complications, including increased risks of stroke, peripheral thromboembolism, heart failure, and symptoms such as shortness of breath, palpitations and fatigue. Medications may be used to treat AFib, but often carry significant side effects and can be ineffective in controlling AFib symptoms and the physiologic sequelae of atrial fibrillation patients. In those instances where medication fails as a treatment method, atrial fibrillation ablation serves as widely-accepted alternative treatment procedure due to its relatively low risk and superior ability to reduce AFib burden. Atrial fibrillation ablation is a type of cardiac ablation in which tissues in the atria are scarred or otherwise destroyed to disrupt faulty electrical signals causing the arrhythmia. These faulty electrical signals are primarily believed to originate from the pulmonary veins near their origination in the left atrium. The most widely-accepted and studied method of treating atrial fibrillation via an ablation procedure is by electrically isolating the pulmonary veins and is referred to as a pulmonary vein isolation (PVI) procedure. PVI procedures may be implemented via radiofrequency procedures (using, for example, catheter or multi-pole catheter tools), ultrasound procedures, or cryoablation or laser ablation procedures (using, for example, balloon tools), each of which aims to create a circumferential, contiguous set of lesions around the (typically) four pulmonary veins in the left atrium. Other areas of the left and right atrium may be additionally targeted, but these techniques vary greatly based on patient characteristics and operator preference. Other energy sources and methods are being developed to more quickly and safely isolate the pulmonary veins, but all result in creating a “ring” of scar around the four pulmonary veins.

The AFib ablation procedure volume over the past 10 years has grown exponentially and advancements in technology have provided for safer and more efficient ablation procedures. For example, use of mapping systems in ablation procedures permit the operator to recreate the left atrium and pulmonary veins on a computer model, which provides for more accurate ablation with reduced fluoroscopy times, and, thus, a reduction in radiation exposure to both the patient and operator. There are also treatment centers that perform AFib ablation using no fluoroscopy at all. This “zero-fluoroscopy” procedure is accomplished via use of intracardiac echocardiography (ICE), which allows for real-time visualization of the heart and its structures. ICE-derived contours of the internal heart structure may then be used to create a 3-D digital representation of the left atrial anatomy using 3-D mapping systems, such as the CARTO® mapping system available through Biosense Webster (Diamond Bar, Calif.).

In view of this increase in AFib ablation procedure volume, including earlier use of ablation procedures in the overall algorithm in the treatment of AFib, there has been considerable research directed to the minimization of complications associated with such procedures. To that end, although the industry has seen a reduction in the complication rate of these procedures, such as a reduced incidence of stroke from aggressive anticoagulation and a reduced incidence of perforation through the use of contact force sensing catheters, there have been few advancements to reduce the incidence of atrioesophageal fistula. Atrioesophageal fistula (AEF) may occur after ablation is performed in any area of the posterior wall of the left atrium that lies in direct contact with the esophagus. There are several mechanisms thought to play a role in the development of AEF, including, most significantly, the proximity of the esophagus to the posterior wall of the left atrium. In an attempt to reduce the likelihood of AEF, operators have sought to modify various aspects of ablation procedures in the posterior left atrium. For example, when ablating areas in the posterior wall of the left atrium thought to lie in contact with or in near proximity to the esophagus, many operators might use a lower power and/or temperature setting for applications involving radiofrequency energy (which may lengthen the duration of the ablation procedure), while other operators might increase the power and ablate for a shorter duration. Temperature probes are also used to assess esophageal heating, and energy delivery may be truncated when a certain temperature threshold is reached or when a particular rate of increase in temperature is detected. In general, all of these techniques essentially decrease the amount of energy delivered in the areas in the posterior wall of the left atrium thought to lie in contact with or in near proximity to the esophagus. While each of these methods seek to avoid or at the very least reduce the potential for esophageal injury, esophageal complications still occur and present a multitude of risks, including life-threatening complications, such as AEF, or, in less severe but more frequent cases, esophageal injury that may lead to gastroparesis, ulceration and/or dysphagia. Moreover, while these techniques aim to reduce the amount of energy delivered to those areas in the posterior wall of the left atrium, there are increased recurrences of AFib post-ablation due to incomplete ablation of atrial tissue. Furthermore, many operators limit energy delivered to the entire posterior wall of left atrium despite the fact that a large portion of the posterior wall may, in fact, be free or sufficiently clear from esophageal contact, thus often leading to under-ablation of the posterior wall.

Some treatment protocols now include the use of esophageal “deviation devices”, which attempt to “clear” or move the esophagus away from the posterior wall of the left atrium both prior to and during the ablation procedure (that is, to deviate the esophagus such that the posterior wall of the left atrium is no longer in contact therewith or in near proximity thereto). These procedures involve identifying the path of the esophagus by injecting into the esophagus iodinated radiocontrast solutions through an orogastric tube in order to visualize the esophagus using fluoroscopy. Using catheter position on fluoroscopy, the operator is able to approximate the location of the esophagus on an anatomical map to guide his or her decision on how best to proceed with ablation. The foregoing location approximation process must be conducted both prior to esophageal deflection and subsequent to deflection in order to verify that the esophagus has been moved away from the area of intended ablation. That process can be rather tedious and may lead to inaccuracies given the difficulty of extrapolating the position of the esophagus on fluoroscopy while simultaneously estimating its position on the anatomical map. Moreover, this process also fails to inform the operator as to what area of the esophagus may be in contact with or in near proximity to the posterior wall of the left atrium, principally due to the 2-D nature of fluoroscopy (which also impacts, if not undercuts, the intent and work flow of zero and low-fluoroscopy labs). Although areas of the esophagus may be visualized using ICE (the ICE-derived contours of which are integrated into the left atrial map), such ICE-facilitated visualizations alone provide only an approximation of esophageal position, often do not effectively serve to differentiate the esophagus from surrounding tissue, and, perhaps most significantly, do not reliably identify the complete girth or width of those areas of the esophagus that lie in contact with or in near proximity to those areas of the posterior wall of the left atrium that the operator intends to ablate.

Therefore, a need exists for compositions, methods and systems for accurately identifying, in real time, the position and/or orientation of the esophagus relative to the left atrium prior to and during atrial fibrillation ablation procedures, so as to avoid or reduce the incidence of AEF. Such compositions, methods and systems would include the identification and visualization of the esophagus, the rapid and accurate integration of the visualized esophagus into an anatomical map together with the posterior wall of the left atrium, in each case presented as a 3-D map, so as to facilitate the accurate identification of those areas of the esophagus that lie in contact with or in near proximity to those areas of the posterior wall of the left atrium that the operator intends to ablate. Such methods, compositions and systems would preferably be conducted with zero or substantially reduced fluoroscopy.

### SUMMARY OF THE INVENTION

The present invention is directed to compositions, methods and systems for identifying the position and/or orientation of the esophagus relative to the left atrium prior to and during atrial fibrillation ablation procedures. Such compositions, methods and systems may be used to avoid or reduce the incidence of AEF.

In an embodiment of the present invention, a composition comprising an echocontrast agent is introduced into the esophagus so as to enhance real-time visualization of the esophagus under intracardiac echocardiography (ICE), and thereby visually differentiate the esophageal lumen from surrounding tissue. Under ICE, the “contrast enhanced” esophagus may then be imaged to generate a 3-D map or digital representation of the esophagus using available 3-D mapping systems, such as the CARTO® mapping system. This 3-D esophageal map, together with the 3-D map of the left atrium (previously imaged via ICE), may then be collectively used to visually and accurately identify the entirety of any area, or the full width of any portion, of the esophagus that lies in contact with or in near proximity to those areas of the posterior wall of the left atrium that the operator intends to ablate. Thereafter, using available esophageal deviation devices, the esophagus may be moved away from any such area of intended ablation (e.g., translated from a first position to a second position that is distinct from the first position), and the esophagus re-imaged and re-mapped and then re-evaluated against the 3-D map of the left atrium to ensure sufficient clearance from the intended area of ablation. Additional amounts of the echocontrast agent may be introduced into the esophagus after deflection to ensure complete visualization of the esophagus prior to ablation. The foregoing composition, method and system may be used to avoid or reduce the incidence of AEF.

The compositions used in the afore-described methods and systems may alternatively comprise: one or more echocontrast agents and one or more viscosity agents; one or more echocontrast agents and one or more carrier solutions; one or more echocontrast agents, one or more viscosity agents and one or more carrier solutions; one or more echocontrast agents and one or more radiocontrast agents; one or more echocontrast agents, one or more radiocontrast agents and one or more carrier solutions; one or more echocontrast agents and one or more coating agents; or one or more echocontrast agents, one or more coating agents and one or more carrier solutions. In each case, the contrast agent(s), when introduced into the esophagus, serve to enhance real-time visualization of the esophagus under appropriate imaging technologies (e.g., ICE-based imaging), and thereby visually differentiate the esophageal lumen from surrounding tissue. Moreover, in each case, the viscosity agents, radiocontrast agents, coating agents and carrier solutions serve to more effectively deliver and coat the entirety or any selected portion of the esophagus with the contrast agent(s). Additionally, and as more fully described herein, while the present invention contemplates use of a radiocontrast agent as a viscosity agent to more effectively deliver and coat the esophagus with the contrast agent(s), use of a radiocontrast agent optionally provides the operator with the ability to view the esophagus as a 2-D structure under fluoroscopy, whether prior to or during the ablation procedure, and to thus receive additional details pertaining to the location and orientation of the esophagus.

These and other features and advantages of the present invention will become apparent to those of ordinary skill in the art after reading the following Detailed Description of the Invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate various embodiments of the present invention and, together with the general description of the invention given above, and the detailed description of the embodiments given below, serve to explain the embodiments of the invention.

FIG. 1 depicts an ICE image of the posterior wall of the left atrium prior to injection of an echocontrast agent into the esophagus (see, orange oval).

FIG. 2 depicts the ICE image of the posterior wall of the left atrium of FIG. 1 and the esophagus after an echocontrast agent has been injected into the esophagus (see, orange oval).

FIG. 3 depicts an ICE image of the posterior wall of the left atrium and the esophagus after an echocontrast agent has been injected into the esophagus, wherein at least a portion of the esophagus has been traced (see, green line) to define an esophageal contour for incorporation into a CARTO® anatomical map.

FIG. 4 depicts the esophageal contour defined in FIG. 3 as incorporated into the CARTO® anatomical map (see, blue arrows pointing to the esophageal contour both in the ICE image and the CARTO anatomical map (oriented in a posterior-anterior view)).

FIG. 5 depicts a completed esophageal map superimposed onto a map of the posterior wall of the left atrium in the CARTO® anatomical map (oriented in a posterior-anterior view) (see, blue arrow pointing to the completed esophageal map shown in light green).

FIG. 6A depicts the position of an ablation catheter shown within fluoroscopic image data of an esophagus enhanced with a radioconstrast agent (e.g., gastrograffin)), and FIG. 6B depicts the echo-contours of the esophagus (enhanced with an echocontrast agent (e.g., the DEFINITY® contrast agent) on the CARTO® anatomical map (in both FIGS. 6A and 6B, a blue oval encircles and identifies the ablation catheter on the right edge of the esophagus, and a blue arrow identifies the esophagus).

FIGS. 7A-7D depict alternate views of the CARTO® anatomical map showing an esophageal map created using echo contours of the esophagus (see, orange arrow identifying the esophageal map in light green; and see, blue arrows identifying the approximate border of the esophagus extrapolated to the CARTO® anatomical map from a fluoroscopic image of the radiocontrast enhanced esophagus of FIG. 6A, showing overall correlation between the fluoroscopic and echo-contour methods.

FIG. 8 depicts esophageal deviation shown within fluoroscopic image data (see, blue arrow identifying an EsoSure device placed into the esophagus and deviating the esophagus to the left).

FIG. 9 depicts esophageal deviation on a CARTO® map, in which an echocontrast agent aids in visualization of the esophagus (see, orange arrow identifying the initial esophageal position (shown in light green), and see the blue arrow identifying the re-mapped esophagus (shown in light purple) after rightward deflection).

### DETAILED DESCRIPTION OF THE INVENTION

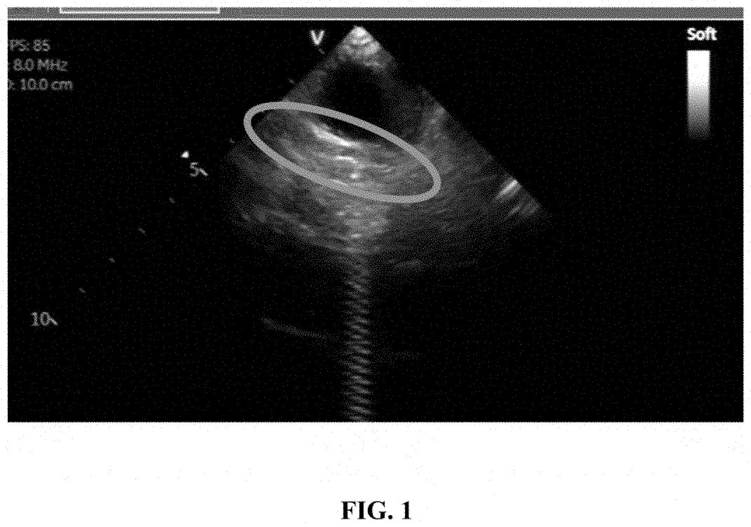

For simplicity and illustrative purposes, the principles of the present invention are described by referring to various exemplary embodiments thereof, and which embodiments may be depicted in FIGS. 1-9. It is understood that the present invention is not limited to the particular examples, embodiments or methods described herein or otherwise depicted in the Figures, as these may vary. It is also to be understood that the terminology used herein is used for the purpose of describing particular embodiments only, and is not intended to limit the scope of the present invention. Moreover, although certain methods may be described with reference to certain steps that are presented herein in a certain order, in many instances, these steps may be performed in any order as would be appreciated by one of ordinary skill in the art, and thus the methods are not limited to the particular arrangement of steps disclosed herein. It must be noted that as used herein and in the appended claims, the singular forms “a”, “an” and “the” include the plural reference unless the context clearly dictates otherwise.

Unless defined otherwise, all technical and scientific terms used herein have the same meanings as commonly understood by one of ordinary skill in the art to which this invention belongs. Specific methods and materials are described, although any methods and materials similar or equivalent to those described herein can be used in the practice or testing of the present invention.

Referring now to FIGS. 1-9, the present invention is directed to compositions, methods and systems for identifying the relative positions and/or orientations (“correspondences”) of the esophagus with respect to the left atrium prior to and during atrial fibrillation ablation procedures. Such compositions, methods and systems may be used to avoid or reduce the incidence of AEF.

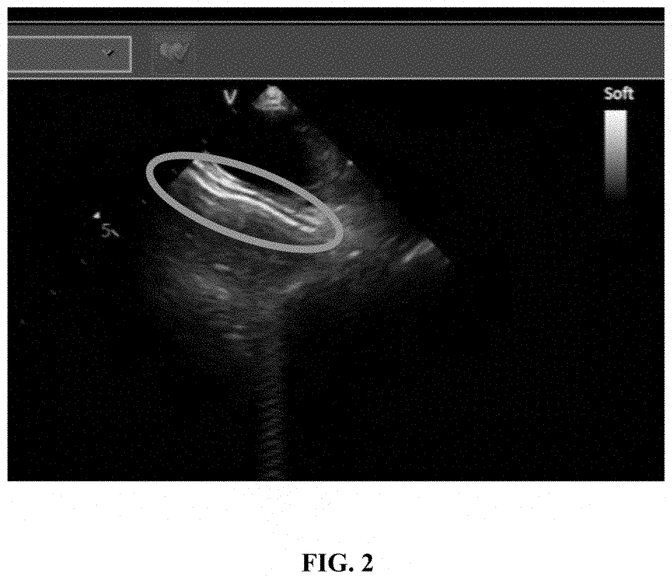

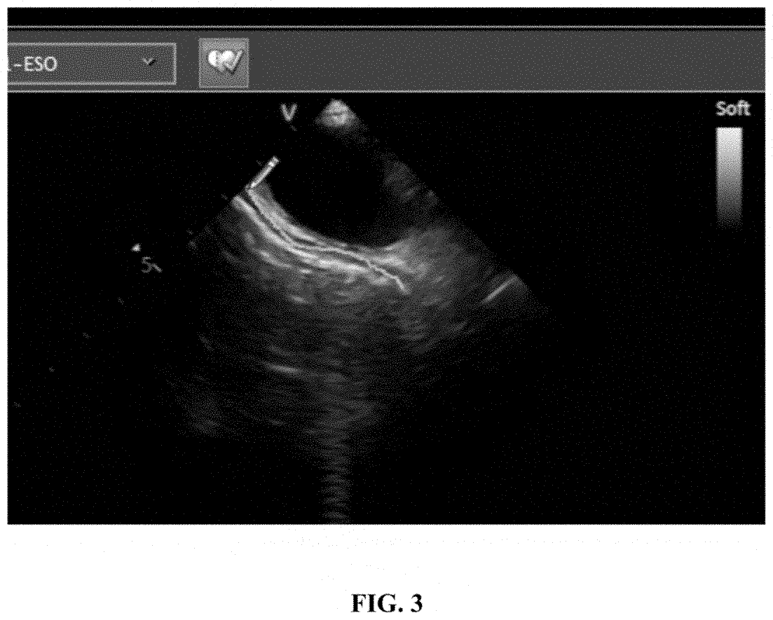

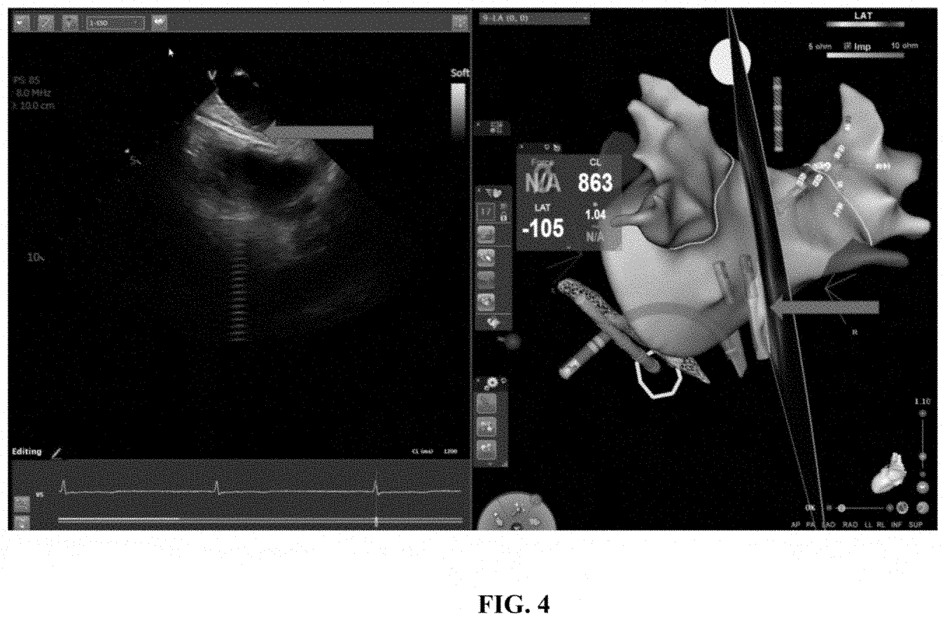

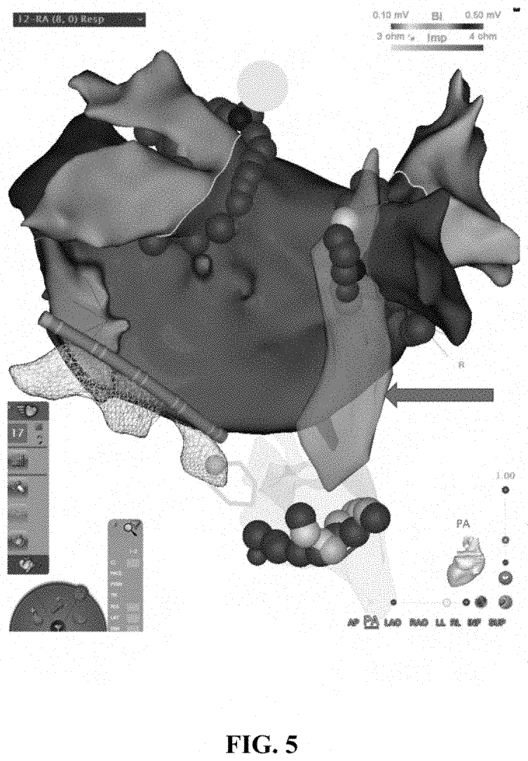

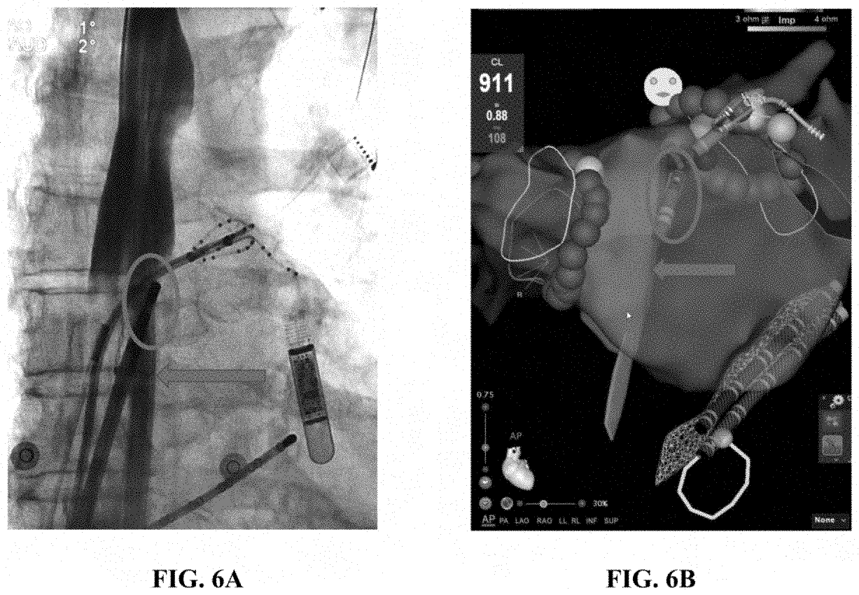

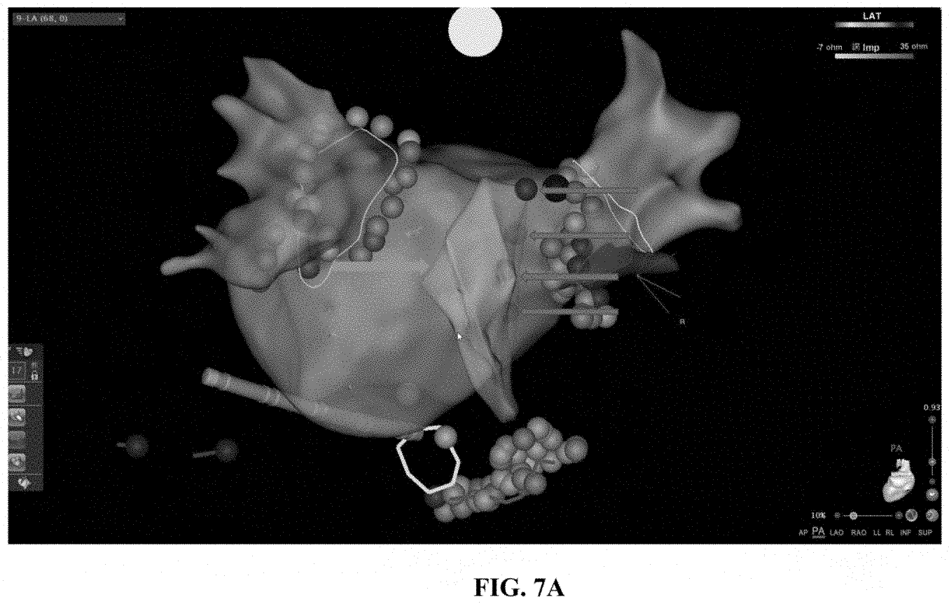

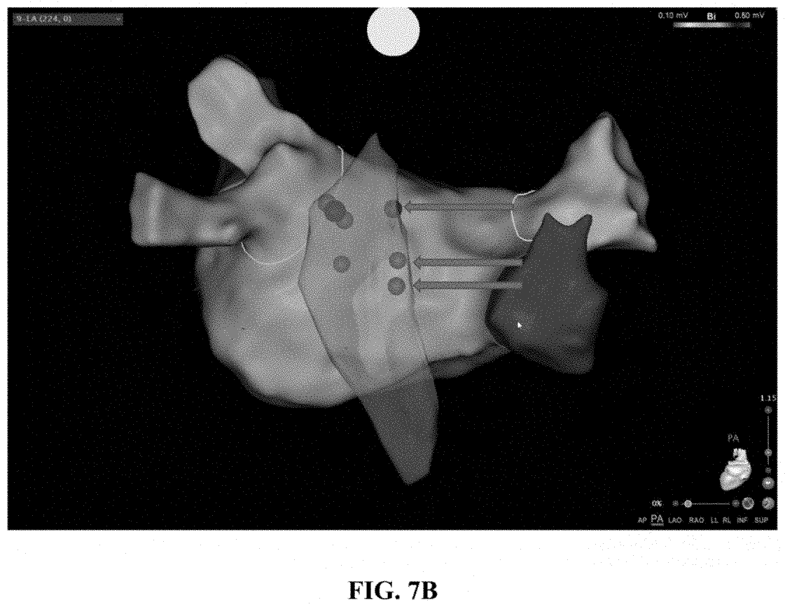

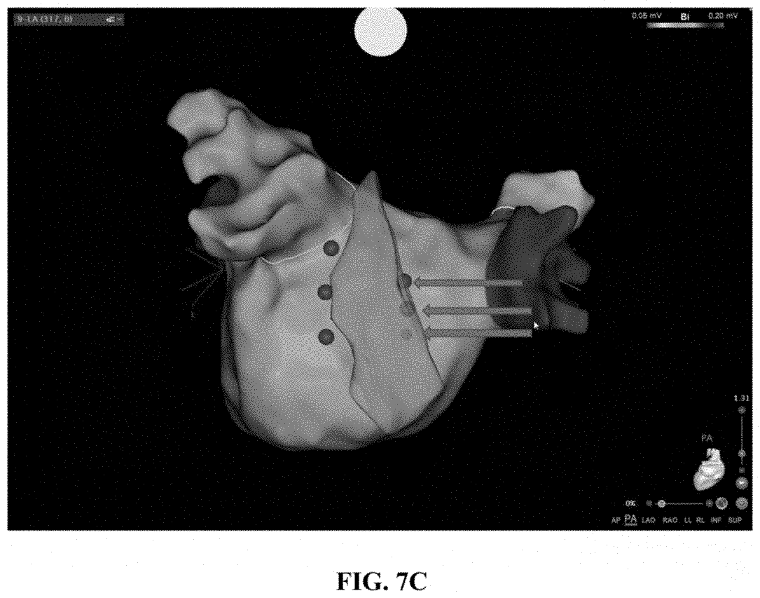

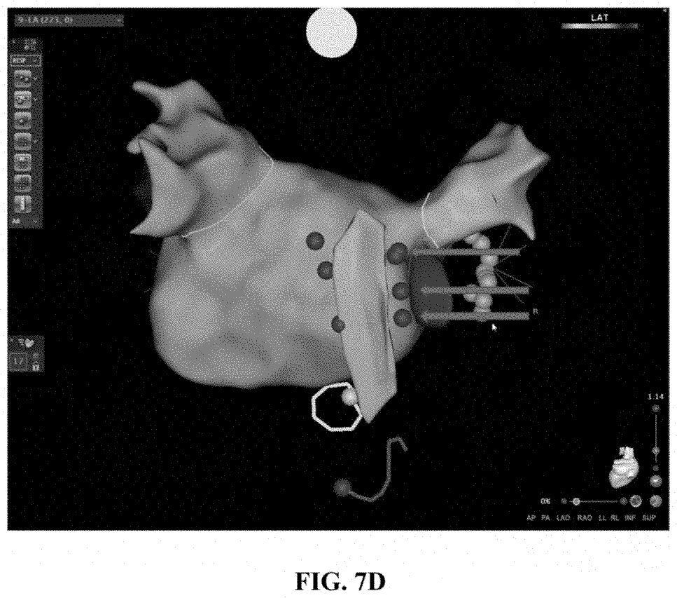

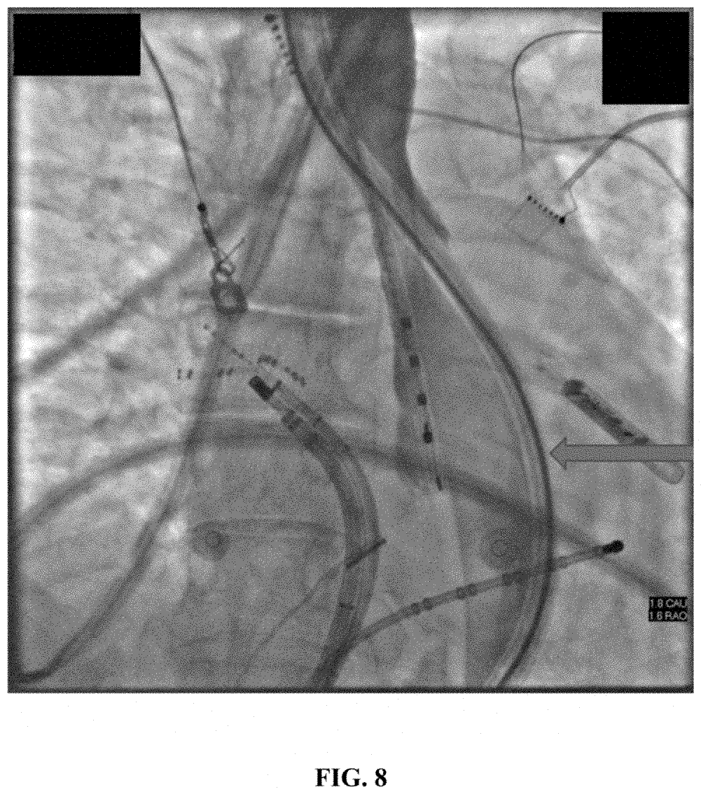

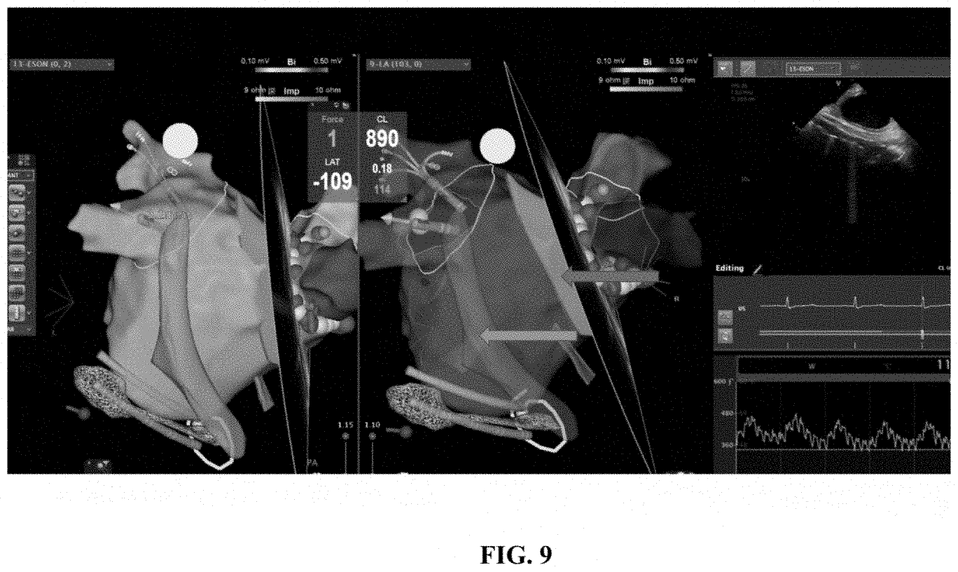

In an embodiment of the present invention, a composition comprising an echocontrast agent is introduced into the esophagus so as to enhance real-time visualization of the esophagus under intracardiac echocardiography (ICE), and thereby visually differentiate the esophageal lumen from surrounding tissue (see, e.g., FIGS. 1, 2). Under ICE, the “contrast enhanced” esophagus is then imaged and traced to generate a contour (see, e.g., FIG. 3) which is then incorporated into a 3-D map or digital representation of the esophagus using available 3-D mapping systems, such as the CARTO® mapping system (see, e.g., FIG. 4). Multiple tracings of sequential esophageal ICE images are taken to yield esophageal contours, which, in the aggregate, will be incorporated into the CARTO® mapping system for the creation of the 3-D map of the esophagus. This 3-D esophageal map, superimposed on and together with the 3-D map of the left atrium (previously imaged via ICE), is then used to visually and accurately identify the entirety of any area, or the full width of any portion, of the esophagus that lies in contact with or in near proximity to those areas of the posterior wall of the left atrium that the operator intends to ablate (see, e.g., FIG. 5). In particular, superimposing the respective 3-D maps (or representations) facilitates a determination of a correspondence between the esophagus and the left atrium. For example, superimposing the respective 3-D maps facilitates a determination of the position and/or orientation of the esophagus relative to the left atrium including, but not limited to, an assessment of the radial distance (i.e., in the “spine to chest” direction) of the posterior wall of the left atrium to the anterior wall of the esophagus, as well as the lateral position (i.e., in the “arm-to-arm” direction) of the esophagus relative to the posterior wall of the left atrium. By adding radiocontrast to the echocontrast as part of the solution injected into the esophagus (as described above), optional cross-validation of the position and/or orientation of the esophagus relative to the left atrium may be achieved by generating fluoroscopic image data via brief fluoroscopy, and by marking or annotating, on the CARTO® map of the left atrium, the esophageal border as seen on the fluoroscopic image data (see, e.g., FIGS. 6A, 6B, and 7A-7D). In an embodiment, the brief fluoroscopy is for a limited duration of five seconds or less, and preferably for a limited duration of one second to two seconds or less, and more preferably for a limited duration of less than one second. Thereafter, using available esophageal deviation devices, the esophagus may be moved away from any such area of intended ablation (see, e.g., FIG. 8), and the esophagus re-imaged and re-mapped and then re-evaluated against the 3-D map of the left atrium to ensure sufficient clearance from the intended area of ablation (see, e.g., FIG. 9).

In the foregoing embodiment of the present invention, it is contemplated that the composition may alternatively comprise: one or more echocontrast agents and one or more viscosity agents; one or more echocontrast agents and one or more carrier solutions; one or more echocontrast agents, one or more viscosity agents and one or more carrier solutions; one or more echocontrast agents and one or more radiocontrast agents; one or more echocontrast agents, one or more radiocontrast agents and one or more carrier solutions; one or more echocontrast agents and one or more coating agents; or one or more echocontrast agents, one or more coating agents and one or more carrier solutions. In each such composition, the echocontrast agent, when introduced into the esophagus, serves to enhance real-time visualization of the esophagus under ICE, and thereby visually differentiate the esophageal lumen from surrounding tissue. Moreover, in each such composition, the viscosity agents, coating agents and carrier solutions serve to more effectively deliver and coat the entirety or any selected portion of the esophagus with the echocontrast agent.

In the foregoing embodiment of the present invention, the echocontrast agent that may be used in the several described compositions includes, for exemplary purposes only, the DEFINITY® contrast agent (available from Lantheus Medical Imaging) and/or the OPTISON™ contrast agent (available from GE Healthcare). The DEFINITY® contrast agent is an injectable ultrasound contrast agent comprised of lipid-coated echogenic microbubbles filled with octafluoropropane gas, and the OPTISON™ contrast agent is a sterile non-pyrogenic suspension of microspheres of human serum albumin with perflutren (i.e., perflutren protein-type A microspheres injectable suspension, USP)). With either of these contrast agents, the microbubbles (in DEFINITY®) and the microspheres (in OPTISON™) create an echogenic contrast effect in the blood. Specifically, the acoustic impedance of the microbubbles/microspheres is much lower than that of the blood. Therefore, impinging ultrasound waves are scattered and reflected at the microbubble/microsphere-blood interface and ultimately may be visualized in the ultrasound image. At the frequencies used in adult echocardiography (2-5 MHz), the microbubble/microspheres resonate which further increases the extent of ultrasound scattering and reflection. The viscosity or coating agent(s) that may be used in the several described compositions include, for exemplary purposes only, one or more of gastrograffin (a radiocontrast agent), sucralfate, barium sulfate, or polyethylene glycol solution. The carrier solution(s) that may be used in the several described compositions include, for exemplary purposes only, one or more of saline, water with dextrose or thickening agent, or other agent to slow motility through the esophagus and coat the esophagus. In the event any one of the several described compositions uses a radiocontrast agent (e.g., gastrograffin) as a viscosity agent, such radiocontrast agent optionally provides the operator with the ability to view the esophagus as a 2-D structure under fluoroscopy, whether prior to or during the ablation procedure, and to thus receive additional details pertaining to the location and orientation of the esophagus.

In another embodiment of the present invention, a composition comprising the DEFINITY® contrast agent (an echocontrast agent), gastrograffin (a radiocontrast agent serving as a viscosity agent) and saline (as a carrier agent), is introduced into the esophagus so as to enhance real-time visualization of the esophagus under intracardiac echocardiography (ICE), and thereby visually differentiate the esophageal lumen from surrounding tissue (see, e.g., FIGS. 1, 2). Under ICE, the “contrast enhanced” esophagus is then imaged and traced to generate a contour (see, e.g., FIG. 3) which is then incorporated into a 3-D map or digital representation of the esophagus using available 3-D mapping systems, such as the CARTO® mapping system (see, e.g., FIG. 4). Multiple tracings of sequential esophageal ICE images are taken to yield esophageal contours, which, in the aggregate, will be incorporated into the CARTO® mapping system for the creation of the 3-D map of the esophagus. This 3-D esophageal map, superimposed on and together with the 3-D map of the left atrium (previously imaged via ICE), is then used to visually and accurately identify the entirety of any area, or the full width of any portion, of the esophagus that lies in contact with or in near proximity to those areas of the posterior wall of the left atrium that the operator intends to ablate (see, e.g., FIG. 5). In particular, superimposing the respective 3-D maps (or representations) facilitates a determination of a correspondence between the esophagus and the left atrium. For example, superimposing the respective 3-D maps facilitates a determination of the position and/or orientation of the esophagus relative to the left atrium including, but not limited to, an assessment of the radial distance (i.e., in the “spine to chest” direction) of the posterior wall of the left atrium to the anterior wall of the esophagus, as well as the lateral position (i.e., in the “arm-to-arm” direction) of the esophagus relative to the posterior wall of the left atrium.

Additionally, in the foregoing embodiment, use of a radiocontrast agent as a viscosity agent in the composition (described above) optionally provides the operator with the ability to view the esophagus as a 2-D structure depicted within fluoroscopic image data generated via brief fluoroscopy, and wherein cross-validation of the position and/or orientation of the esophagus relative to the left atrium may be achieved by marking or annotating, on the CARTO® map of the left atrium, the esophageal border as seen on the fluoroscopic image data (see, e.g., FIGS. 6A, 6B, and 7A-7D). In an embodiment, the brief fluoroscopy is for a limited duration of five seconds or less, and preferably for a limited duration of one second to two seconds or less, and more preferably for a limited duration of less than one second. Thereafter, using available esophageal deviation devices, the esophagus may be moved away from any such area of intended ablation (see, e.g., FIG. 8), and the esophagus re-imaged and re-mapped and then re-evaluated against the 3-D map of the left atrium to ensure sufficient clearance from the intended area of ablation (see, e.g., FIG. 9). In the foregoing embodiment, use of the radiocontrast agent gastrograffin as a viscosity agent in the composition, optionally provides the operator with the ability to view the esophagus as a 2-D structure under fluoroscopy, whether prior to or during the ablation procedure, and to thus receive additional details pertaining to the location and orientation of the esophagus.

In yet another embodiment of the present invention, a composition comprising 5 cc of the DEFINITY® contrast agent (an echocontrast agent), 10 cc of gastrograffin (a radiocontrast agent serving as a viscosity agent) and 5 cc of saline (as a carrier agent), is prepared for introduction into the (mid) esophagus (again, for purposes of enhancing visualization of the esophagus under intracardiac echocardiography (ICE), and thereby visually differentiating the esophageal lumen from surrounding tissue) (see, e.g., FIGS. 1, 2). Specifically, the composition is prepared by first mixing 5 cc of the DEFINITY® contrast agent with 5 cc of saline (medium), with that mixture then being drawn into a syringe containing 10 cc of gastrograffin and thus admixed therewith. A standard orogastric tube (e.g., as available from Bard Medical) is advanced through the esophagus and into the stomach of the patient, with gastric contents returned to eliminate the possibility of tracheal placement. The tube is generally pulled back to about 35 cm at the lips, which generally places the open ports of the orogastric tube at the middle portion of the cardiac silhouette—a position that may be verified with brief fluoroscopy in view of radiolucent markers on the orogastric tube. In an embodiment, the brief fluoroscopy is for a limited duration of five seconds or less, and preferably for a limited duration of one second to two seconds or less, and more preferably for a limited duration of less than one second. This brief visualization also identifies the general position of the esophagus; that is, whether the esophagus is more prone to the left or right side of the cardiac silhouette. The composition (contained in the syringe) is then injected into the orogastric tube, the end of which tube is held several feet above the level of the supine patient so as to encourage the composition to flow down into the esophagus. The composition may generally take about 30 seconds to completely coat the esophagus. Once coated with the composition, the “contrast enhanced” esophagus is then imaged under ICE and mapped (using available 3-D mapping systems, such as the CARTO® mapping system) to generate a 3-D map or digital representation of the esophagus, thereby identifying any critical or relevant area of the esophagus—i.e., any area of the esophagus that lies in contact with or in near proximity to those areas of the posterior wall of the left atrium (previously imaged via ICE) that the operator intends to ablate (see, e.g., FIGS. 3, 4).

With specific regard to this imaging process, if the esophagus is closer to the right pulmonary veins, the esophagus is best identified via ICE with the catheter placed in the mid-right atrium. If, however, the esophagus is closer to the left pulmonary veins, the ICE catheter may be deflected into the right ventricular outflow tract, and then rotated clockwise until the esophagus can be visualized via ICE. In either case, with the ICE catheter positioned or otherwise generally directed toward the esophageal structure, and using the CARTO mapping system, the image of the “contrast enhanced” esophagus can be mapped or “traced” along the posterior wall of the left atrium to generate sequential contours, which, in the aggregate, are used to create a 3-D map of the esophagus, which is then added to the left atrial map (again, the left atrium having been previously imaged via ICE) (see, e.g., FIGS. 1-4). This 3-D esophageal map, superimposed on and together with the 3-D left atrial map, is then used to visually and accurately identify the entirety of any area, or the full width of any portion, of the esophagus that lies in contact with or in near proximity to those areas of the posterior wall of the left atrium that the operator intends to ablate (see, e.g., FIG. 5). In particular, superimposing the respective 3-D maps (or representations) facilitates a determination of a correspondence between the esophagus and the left atrium. For example, superimposing the respective 3-D maps facilitates a determination of the position and/or orientation of the esophagus relative to the left atrium including, but not limited to, an assessment of the radial distance (i.e., in the “spine to chest” direction) of the posterior wall of the left atrium to the anterior wall of the esophagus, as well as the lateral position (i.e., in the “arm-to-arm” direction) of the esophagus relative to the posterior wall of the left atrium. It is contemplated herein that the foregoing composition may be used in any “automatic” mapping process that (simultaneously or near simultaneously) maps the esophagus during mapping of the left atrium, and wherein the computer mapping system would identify the “contrast enhanced” esophageal segments and automatically generate a 3-D map of the esophagus superimposed on the 3-D left atrial map.

Following this mapping process, and thus upon determining the position and/or orientation of the esophagus relative to the posterior wall of the left atrium, the esophagus may be moved away from any area of intended ablation (e.g., translated from a first position to a second position that is distinct from the first position) using available esophageal deviation devices (see, e.g., FIG. 8), and the esophagus re-imaged and re-mapped and then re-evaluated against the left atrial map to ensure sufficient clearance from the intended area of ablation. In particular, the “trailing edge” (and, optionally the “leading edge”) of the esophagus is re-imaged and re-mapped to ensure that the esophagus is no longer in contact with or in near proximity to any area of the posterior wall of the left atrium that the operator intends to ablate (see, e.g., FIG. 9). Esophageal deviation devices may include, for example, the EsoSure® esophageal retratactor (available from EPreward, Inc.), or any other physical instrument(s) that “clear” or move the esophagus away from the posterior wall of the left atrium both prior to and during the ablation procedure (that is, deviate the esophagus such that the posterior wall of the left atrium is no longer in contact therewith or in near proximity thereto). Additionally, in the foregoing embodiment, use of a radiocontrast agent, such as gastrograffin, as a viscosity agent in the composition optionally provides the operator with the ability to view the esophagus as a 2-D structure under fluoroscopy, and wherein cross-validation of the position and/or orientation of the esophagus may be achieved by marking or annotating, on the CARTO® map of the left atrium, the esophageal border as seen on fluoroscopy during either the initial mapping or re-mapping processes described hereinabove (see, e.g., FIGS. 6A, 6B, and 7A-7D).

It is contemplated herein that, while certain aspects of the foregoing systems and methods may use fluoroscopy (for instance, at the initial stages of inserting and positioning the orogastric tube prior to injection of the composition, as described above), the present systems and methods may entirely dispense with fluoroscopy through use of improved orogastric tube designs. For example, an orogastric tube, with integral channels through which sensor-based or electrode-based catheters may be fed, would be visible under ICE, thus entirely dispensing with any fluoroscopy, whether at the afore-described initial stages of inserting and positioning the orogastric tube, or otherwise. Moreover, to facilitate a “contrast enhanced” esophageal visualization throughout the ablation procedure, an improved orogastric tube design with one or more exit holes on the side of the tube would allow for multiple injections of contrast agent, whether prior to or subsequent to esophageal deviation, so as to respond to any contrast agent prematurely draining into the stomach.

In each of the embodiments described herein, it is contemplated that catheter-based contact mapping may be used in addition to, or as an alternative to, 3-D mapping of the left atrium derived from ICE-based images. Moreover, 3-D mapping systems for cardiac ablation other than CARTO® mapping may be developed in the future that incorporate ultrasound-based 3-D mapping that would be able to incorporate the aforementioned techniques to enhance esophageal visualization and incorporation into an anatomical map.

Upon completion of the ablation procedure, the esophageal deviation device, if used, is removed, and the orogastric tube is removed from the stomach through the esophagus under continuous suction to remove any residual composition to minimize the possibility of aspiration during extubation and recovery.

As described herein, compositions of the present invention may be formulated to include a contrast agent, a viscosity or coating agent, and a carrier agent, which, collectively, function to deliver and coat the esophagus. As such, alternate compositions may include: 5 cc of the DEFINITY® contrast agent (as the echocontrast agent), 10 cc of sucralfate solution (as a viscosity agent) and 5 cc of saline (as a carrier agent); 5 cc of the DEFINITY® contrast agent (as the echocontrast agent), 10 cc of barium sulfate (as a viscosity agent) and 5 cc of saline (as a carrier agent); and, 5 cc of the DEFINITY® contrast agent (as the echocontrast agent), 10 cc of polyethylene glycol solution (as a viscosity agent) and 5 cc of saline (as a carrier agent).

As described herein, a method for implementing the present invention may include introducing a composition into an esophagus. An image of the esophagus is obtained using a sensor disposed within a portion of a heart proximate to the esophagus. A three-dimensional (“3-D”) esophageal representation is created using the image. The 3-D esophageal representation is superimposed onto a 3-D left atrial representation. A correspondence is determined between the 3-D esophageal representation and the 3-D left atrial representation. In an embodiment, the composition comprises an echocontrast agent. In an embodiment, the composition further comprises a radiocontrast agent. In an embodiment, generating the 3-D esophageal representation comprises tracing at least a portion of the esophagus within the image to define an esophageal contour. In an embodiment, the correspondence is a radial distance between a posterior wall of the left atrium of the heart and the anterior wall of the esophagus, a lateral position of the esophagus relative to the posterior wall of the left atrium, or a combination thereof. In an embodiment, obtaining the image comprises directing ultrasonic energy towards the esophagus via the left atrium wall of the heart. In an embodiment, the composition comprises: one or more echocontrast agents and one or more viscosity agents; one or more echocontrast agents and one or more carrier solutions; one or more echocontrast agents, one or more viscosity agents and one or more carrier solutions; one or more echocontrast agents and one or more radiocontrast agents; one or more echocontrast agents, one or more radiocontrast agents and one or more carrier solutions; one or more echocontrast agents and one or more coating agents; or one or more echocontrast agents, one or more coating agents and one or more carrier solutions.

In an embodiment, the method further includes introducing a radiocontrast agent into the esophagus, and obtaining a fluoroscopic image of the esophagus. In an embodiment, the method further includes obtaining an additional image of the esophagus using the sensor subsequent to the esophagus being translated from a first position to a second position distinct from the first position, updating the 3-D esophageal representation using the additional image, and determining an updated correspondence between the 3-D esophageal representation and the 3-D left atrial representation. In an embodiment, the method further includes dynamically updating the 3-D esophageal representation in real-time as additional image data is obtained using the sensor. In an embodiment, the method further includes obtaining fluoroscopic image data of the esophagus enhanced with a radiocontrast agent, and validating the correspondence between the 3-D esophageal representation and the 3-D left atrial representation using the fluoroscopic image data. In an embodiment, the fluoroscopic image data is generated via fluoroscopy of a limited duration. In an embodiment, the limited duration is of five seconds or less, and preferably of one second to two seconds or less, and more preferably less than one second.

As described herein, a system for implementing the present invention may include a sensor, a processor, and a computer-readable storage medium comprising instructions. Upon execution by the processor, the instructions cause the system to perform operations. The operations include obtaining an image of an esophagus injected with a composition while the sensor is disposed within a portion of a heart proximate to the esophagus. A three-dimensional (“3-D”) esophageal representation is created using the image. The 3-D esophageal representation is superimposed onto a 3-D left atrial representation. A correspondence is determined between the 3-D esophageal representation and the 3-D left atrial representation. In an embodiment, obtaining the image comprises directing ultrasonic energy towards the esophagus via the left atrium wall of the heart. In an embodiment, the correspondence is a radial distance between a posterior wall of the left atrium of the heart and the anterior wall of the esophagus, a lateral position of the esophagus relative to the posterior wall of the left atrium, or a combination thereof.

In an embodiment, the instructions, when executed, further cause the system to perform additional operations comprising dynamically updating the 3-D esophageal representation in real-time as additional image data is obtained using the sensor. In an embodiment, the instructions, when executed, further cause the system to perform additional operations comprising obtaining fluoroscopic image data of the esophagus enhanced with a radiocontrast agent, and validating the correspondence between the 3-D esophageal representation and the 3-D left atrial representation using the fluoroscopic image data. In an embodiment, the fluoroscopic image data is generated via fluoroscopy of a limited duration. In an embodiment, the limited duration is of five seconds or less, and preferably of one second to two seconds or less, and more preferably less than one second. In an embodiment, the instructions, when executed, further cause the system to perform additional operations comprising obtaining an additional image of the esophagus using the sensor subsequent to the esophagus being translated from a first position to a second position distinct from the first position, updating the 3-D esophageal representation using the additional image, and determining an updated correspondence between the 3-D esophageal representation and the 3-D left atrial representation.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of structures and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A method comprising:
introducing a composition into an esophagus;
obtaining an image of said esophagus using a sensor disposed within a portion of a heart proximate to said esophagus;
creating a three-dimensional (“3-D”) esophageal representation using said image;
superimposing said 3-D esophageal representation onto a 3-D left atrial representation; and
determining a correspondence between said 3-D esophageal representation and said 3-D left atrial representation.

2. The method of claim 1, wherein said composition comprises an echocontrast agent.

3. The method of claim 2, wherein said composition further comprises a radiocontrast agent.

4. The method of claim 1, wherein generating said 3-D esophageal representation comprises:
tracing at least a portion of said esophagus within said image to define an esophageal contour.

5. The method of claim 1, wherein said correspondence is a radial distance between a posterior wall of a left atrium of said heart and an anterior wall of said esophagus, a lateral position of said esophagus relative to said posterior wall of said left atrium, or a combination thereof.

6. The method of claim 1, wherein obtaining said image comprises:
directing ultrasonic energy towards the esophagus via a left atrium wall of said heart.

7. The method of claim 1, further comprising:
introducing a radiocontrast agent into said esophagus; and
obtaining a fluoroscopic image of said esophagus.

8. The method of claim 1, further comprising:
obtaining an additional image of said esophagus using said sensor subsequent to said esophagus being translated from a first position to a second position distinct from said first position;
updating said 3-D esophageal representation using said additional image; and
determining an updated correspondence between said 3-D esophageal representation and said 3-D left atrial representation.

9. The method of claim 1, further comprising:
dynamically updating said 3-D esophageal representation in real-time as additional image data is obtained using said sensor.

10. The method of claim 1, further comprising:
obtaining fluoroscopic image data of said esophagus enhanced with a radiocontrast agent; and
validating said correspondence between said 3-D esophageal representation and said 3-D left atrial representation using said fluoroscopic image data.

11. The method of claim 10, wherein said fluoroscopic image data is generated via fluoroscopy of a duration selected from the group consisting of: five seconds or less, one second to two seconds or less, and less than one second.

12. The method of claim 11, wherein said composition comprises: one or more echocontrast agents and one or more viscosity agents; one or more echocontrast agents and one or more carrier solutions; one or more echocontrast agents, one or more viscosity agents and one or more carrier solutions; one or more echocontrast agents and one or more radiocontrast agents; one or more echocontrast agents, one or more radiocontrast agents and one or more carrier solutions; one or more echocontrast agents and one or more coating agents; or one or more echocontrast agents, one or more coating agents and one or more carrier solutions.

13. A system comprising:
a sensor;
a processor; and
a computer-readable storage medium comprising instructions that, upon execution by the processor, cause the system to perform operations, the operations comprising:
obtaining an image of an esophagus injected with a composition while said sensor is disposed within a portion of a heart proximate to said esophagus;
creating a three-dimensional (“3-D”) esophageal representation using said image;
superimposing said 3-D esophageal representation onto a 3-D left atrial representation; and
determining a correspondence between said 3-D esophageal representation and said 3-D left atrial representation.

14. The system of claim 13, wherein said instructions, when executed, further cause said system to perform additional operations, said additional operations comprising:
dynamically updating said 3-D esophageal representation in real-time as additional image data is obtained using said sensor.

15. The system of claim 13, wherein said instructions, when executed, further cause said system to perform additional operations, said additional operations comprising:
obtaining fluoroscopic image data of said esophagus enhanced with a radiocontrast agent; and
validating said correspondence between said 3-D esophageal representation and said 3-D left atrial representation using said fluoroscopic image data.

16. The system of claim 15, wherein said fluoroscopic image data is generated via fluoroscopy of a duration selected from the group consisting of: five seconds or less, one second to two seconds or less, and less than one second.

17. The system of claim 13, wherein said instructions, when executed, further cause said system to perform additional operations, said additional operations comprising:
obtaining an additional image of said esophagus using said sensor subsequent to said esophagus being translated from a first position to a second position distinct from said first position;
updating said 3-D esophageal representation using said additional image; and
determining an updated correspondence between said 3-D esophageal representation and said 3-D left atrial representation.

18. The system of claim 13, wherein obtaining said image comprises:
directing ultrasonic energy towards the esophagus via a left atrium wall of said heart.

19. The system of claim 13, wherein said correspondence is a radial distance between a posterior wall of a left atrium of said heart and an anterior wall of said esophagus, a lateral position of said esophagus relative to said posterior wall of said left atrium, or a combination thereof.

20. A composition for enhancing real-time visualization of an esophagus, said composition comprising: one or more echocontrast agents and one or more viscosity agents; one or more echocontrast agents and one or more carrier solutions; one or more echocontrast agents, one or more viscosity agents and one or more carrier solutions; one or more echocontrast agents and one or more radiocontrast agents; one or more echocontrast agents, one or more radiocontrast agents and one or more carrier solutions; one or more echocontrast agents and one or more coating agents; or one or more echocontrast agents, one or more coating agents and one or more carrier solutions.
